(19) United States
(12) Patent Application Publication
(10) Pub. No.: US 2023/0146392 A1
(43) Pub. Date: May 11, 2023

(54) LOW-FRICTION COATINGS FOR SYRINGES AND RELATED METHODS

(71) Applicant: Merit Medical Systems, Inc., South Jordan, UT (US)

(72) Inventors: Jim Mottola, West Jordan, UT (US); Richard P. Jenkins, Bluffdale, UT (US); Gregory R. McArthur, Sandy, UT (US)

(21) Appl. No.: 18/053,953

(22) Filed: Nov. 9, 2022

Related U.S. Application Data
(60) Provisional application No. 63/263,851, filed on Nov. 10, 2021.

(51) Int. Cl. A61M 5/31; C08L 29/04; C09D 129/04

(52) U.S. Cl. CPC A61M 5/3129; C08L 29/04; C09D 129/04; A61M 2005/3131; C08L 2203/02

(57) ABSTRACT

Coatings for syringe plunger tips, as well as syringes and syringe components including such coatings, are disclosed. A material of the coating may have at least one different material property than a material of the syringe component to which it is applied. The coatings may provide a reduced coefficient of friction between the coating and a surface of the syringe relative to the coefficient of friction between the uncoated component and the syringe surface. Methods for manufacturing a syringe or syringe component including such coatings, as well as methods for isolating a substance in a syringe from a syringe component, are also disclosed.

### RELATED APPLICATIONS

This application claims priority to U.S. Provisional Application No. 63/263,851, filed on Nov. 10, 2021 and titled, “Low-Frication Coatings for Syringes and Related Methods,” which is hereby incorporated by reference in its entirety.

### TECHNICAL FIELD

The present disclosure relates to the field of medical devices. More specifically, the present disclosure relates to a syringe having a syringe plunger and a syringe plunger tip. Even more specifically, the present disclosure relates to low-friction coatings for syringes tips and methods related thereto.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings, in which:

FIG. 1A illustrates a perspective view of an embodiment of a syringe assembly according to the present disclosure.

FIG. 1B illustrates a side cross-sectional view of the syringe assembly of FIG. 1A.

FIG. 2 illustrates a side cross-sectional view of an embodiment of a syringe plunger tip according to an embodiment of the present disclosure.

FIG. 3 illustrates a side cross-sectional view of an embodiment of a syringe plunger tip according to an embodiment of the present disclosure.

FIG. 4 illustrates a side cross-sectional view of an embodiment of a syringe plunger tip according to an embodiment of the present disclosure.

FIG. 5A illustrates a side cross-sectional view of a syringe plunger tip according to an embodiment of the present disclosure.

FIG. 5B illustrates a side cross-sectional view of a syringe plunger tip according to an embodiment of the present disclosure.

FIG. 6 illustrates a side cross-sectional view of an embodiment of a syringe assembly according to the present disclosure.

### DETAILED DESCRIPTION

Syringes may be configured to displace fluid via displacement of a plunger tip within a syringe barrel, as described below. Smooth or even displacement of the plunger tip may be beneficial for controlling the rate at which material is dispensed from a syringe. However, compressive forces acting between the syringe tip and an inside surface of a syringe barrel may result in frictional forces that resist displacement of the plunger tip within the syringe barrel. Differences between static and dynamic frictional forces may make it difficult to move a syringe plunger by a small amount, as the plunger may tend to “stick” in a stationary position, then “jump” or suddenly slide quickly as it is displaced. As detailed herein, reduction of friction may facilitate smooth operation of a syringe.

Low-friction coatings for syringes, as well as syringes and syringe components having such coatings, are disclosed herein. In some embodiments, a coating is disposed on at least a portion of an external surface of a syringe plunger tip. The coating may comprise a material that is different than the material of the plunger tip. The material properties of the coating and the plunger tip may differ in at least one material property, as further described below. For example, the coating may comprise a material with a lower coefficient of friction when in contact with the interior surface of a syringe barrel than the material of the plunger tip. Further, in some embodiments, the plunger tip may compress to provide a sealing force between the plunger tip and coating and the interior surface of a syringe barrel.

In some embodiments, the coating may be disposed on one or more portions of the external surface of the plunger tip and may further be substantially restricted to said portions. For example, a plunger tip may comprise an annular wall and may include one or more ridges extending radially from the annular wall, and the coating may be disposed on all or part of the external surface of one or more of the ridges.

In some embodiments, it may be desirable to separate or isolate the material of a plunger tip and/or syringe barrel from the contents of the syringe reservoir. In some such instances, a coating may configured to prevent or minimize contact and/or chemical leaching of materials between a plunger tip and/or syringe barrel and material within a syringe reservoir. For example, in some embodiments it may be desirable to isolate a silicone plunger tip and, for example, a substance containing polyvinyl alcohol (PVA) within the syringe barrel. In another example, a coating may be configured to prevent contact between a silicone plunger tip and a substance containing gelatin foam (examples include SurgiFoam™ from Ethicon or Gelfoam™ from Pfizer) within the syringe barrel. In some instances, silicone plunger tips may be incompatible with use of PVA or gelatin foam. In some instances, for example, silicone material from the plunger tip or silicone lubrication within the syringe barrel may tend to coat or otherwise adhere to the PVA or gelatin foam if these components are in contact. This may, in turn, interfere with hydration of PVA or gelatin foam particles thus causing them to agglomerate. Thus, the coatings provided herein may be impermeable or demonstrate low permeability to silicone to prevent such contamination. As used herein, “silicone” refers broadly to polymeric compounds that consist of silicon-oxygen backbone chains ( . . . —Si—O—Si—O— . . . )ₙ (e.g., compounds containing one or more siloxane groups).

In some embodiments, a coating is disposed on at least a portion of an interior surface of a syringe barrel. The material properties of the materials used for the coating and the interior surface may differ in certain respects. For example, the coating may comprise a material that provides a lower coefficient of friction between the plunger tip and the coating than the coefficient of friction between the plunger tip and the material of the interior surface without the coating.

Certain plunger tips within the scope of this disclosure include one or more sealing rings disposed along an annular wall of the plunger tip. One or more of these sealing rings may be configured to compress and/or deform against an inside surface of the syringe barrel, creating a pressure seal. Thus, the sealing rings may be configured to function as o-rings or sealing portions. As discussed herein, embodiments with one, two, three, four, five, six, or more sealing rings on a single plunger tip are within the scope of this disclosure. Further, one or more annular recesses may be disposed adjacent at least one of the sealing rings. Annular recesses may be configured to provide space to accommodate deformation of the sealing ring to enhance sealing.

The compression, forces, and interaction between the sealing rings and the inside surface of the syringe barrel may be such that a significant proportion of the frictional engagement between the plunger tip and the syringe barrel is at the points of contact between the sealing rings and the syringe barrel. In some instances, a low friction coating may be applied only to the sealing rings, or only to a radially outward-most portion of one or more sealing rings of a plunger tip.

Embodiments within the scope of this disclosure include embodiments wherein a coating is disposed on a portion of an inside surface of a barrel and no coating is disposed on the syringe tip, embodiments wherein a coating is disposed on the syringe tip and no coating is disposed on the barrel, and embodiments wherein both the syringe tip and the barrel comprise coatings. In embodiments wherein both the syringe barrel and plunger tip comprise a coating the coefficient of friction between the two coatings may be configured to facilitate smooth operation of the syringe.

In some embodiments, a coating comprises one or more polymeric materials. Coatings within the scope of this disclosure may comprise polymeric materials including, but not limited to, polyolefins, polyethylene, perfluoropolyether (PFPE), perfluoropolyalkylether (PFAE), or a thermoplastic elastomer. In some embodiments, the coating may comprise a polyethylene including, but not limited to, linear low-density polyethylene (LLDPE), high-density polyethylene (HDPE), expanded polytetrafluoroethylene (ePTFE), polytetrafluoroethylene (PTFE), or a composite of any of these materials. In some embodiments, the coating material may be adhered to, e.g, covalently bonded to, the material of the surface on which it is disposed. Such coatings may facilitate, for example, controlled sliding of a syringe plunger tip along the length of an interior surface of a syringe barrel. Additionally or alternatively, such coatings may prevent a substance loaded within a syringe barrel from being contaminated by, or contaminating, syringe components.

Methods for manufacturing a syringe assembly including coatings for components thereof are also provided by the present disclosure. In some embodiments, the methods comprise applying a coating to a syringe plunger tip, wherein the plunger tip comprises a first material and the coating comprises a second material, and wherein at least one material property of the first material differs from at least one material property of the second material. The coating material may be deposited onto the external surface of the plunger tip through a number of different processes, including, for example, dip coating, sputtering, spray coating, or spin coating. In some embodiments, the method may further comprise loading a substance comprising PVA or gelatin foam into a reservoir defined by an interior surface of a syringe barrel, wherein said reservoir is configured to receive the plunger tip. In some embodiments, a method of manufacturing a syringe can comprise depositing a coating onto a portion of the interior surface of a syringe barrel.

In some embodiments, the coatings described herein may comprise a material that isolates, for example by physical and/or chemical separation, a syringe component from a substance loaded into a syringe barrel. The component may be a plunger tip and/or the interior surface of the barrel itself. Again, this isolation may be due to prevention of physical contact between the component and the substance. Further, the coating may be substantially impermeable to migration of chemical components across the coating.

It will be readily understood with the aid of the present disclosure that the components of the embodiments, as generally described and illustrated in the figures herein could be arranged and designed in a variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The terms “proximal” and “distal” refer to opposite ends of a medical device. As used herein, the proximal end of a medical device is the end nearest a practitioner during use, while the distal end is the opposite end. For example, the proximal end of a standard syringe refers to the end associated with the plunger handle while the distal end corresponds to the outlet.

The term “material property” refers to any property or characteristic of a substance comprising components described herein. For example, material properties may include, but are not limited to, a material's chemical make-up (e.g., materials that contain silicon versus those that do not), a material's coefficient of friction as measured with another material or surface, a material's permeability, and other physical, chemical, mechanical, structural, electrical or other properties.

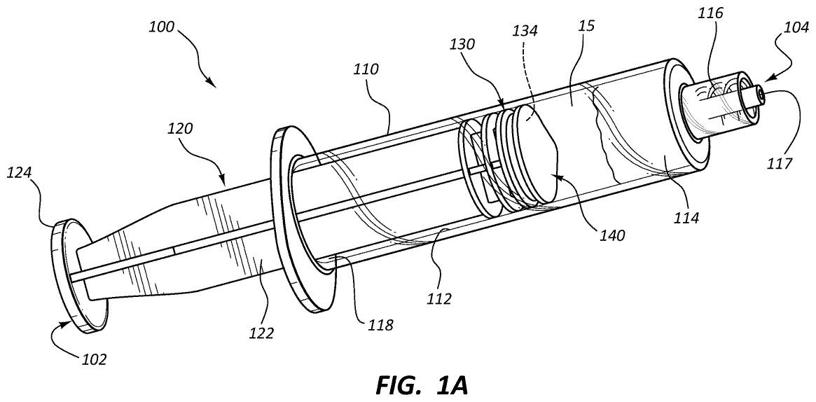

FIGS. 1A and 1B are, respectively, a perspective view and a cross-sectional view of one embodiment of a syringe assembly 100 according to the present disclosure. In the illustrated embodiment, the syringe assembly 100 extends between a proximal end 102 and a distal end 104. Further, the illustrated syringe assembly 100 includes a syringe barrel 110 having an interior surface 112. The interior surface 112 may define a reservoir 114. The reservoir 114 defined by interior surface 112 may be any suitable size and shape, e.g., for receiving a syringe plunger (such as plunger 120 discussed below). In one embodiment, the reservoir 114 may be generally cylindrical in shape. The reservoir 114 defined by interior surface 112 may be configured to receive a substance 15. Syringe barrel 110 may further include a tip 116 having an opening 117 in communication with the reservoir for receiving or expelling the substance 15 from the reservoir 114. In the illustrated embodiment, the tip 116 and opening 117 are disposed adjacent the distal end of the syringe barrel.

Syringe barrel 110 may include an additional opening 118 located, for example, at a proximal end of the barrel, for receiving a plunger 120, which may be disposed or partially disposed within the syringe barrel 110. The plunger 120 may comprise an elongate handle portion 122 having both a proximal end 124 and a distal end 126. The plunger 120 may typically have a length that is at least as long as a length of the syringe barrel 110. Accordingly, the proximal end 124 of the plunger may extend out from the barrel 110 when the plunger is disposed within the barrel 110. The plunger 120 may otherwise have any size and shape that is suitable for being disposed within the syringe barrel 110.

A plunger tip 130 may be coupled to handle portion 122. The plunger tip 130 may be a tip that is integrally molded with handle portion 122 or a tip that is attached separately. In one embodiment, the plunger tip 130 may be coupled to the distal end 126 of handle portion 122. The plunger tip 130 may comprise an external surface 134. In certain embodiments, the plunger tip 130 may be configured to contact at least a portion of the interior surface 112 of barrel 110. Accordingly, the plunger tip 130 may have a size and shape that is complimentary to the size and shape of the reservoir 114 defined by interior surface 112. For example, the plunger tip 130 may be generally cylindrical in shape such that a portion of its external surface 134 may have a circumference that is the same or similar in size to a circumference of the interior surface 112 of a barrel 110 that is also cylindrical in shape.

Furthermore, the plunger tip 130 may be sized such that, when disposed within the syringe barrel 110, the syringe barrel 110 radially constrains and/or compresses the plunger tip 130. The plunger tip 130 may be comprised of an elastomeric material that, due to the compression, provides a radially outward-oriented force on the interior surface 112 of the syringe barrel 110. Compression of the plunger tip 130 may thus result in a radial outward force that tends to seal the plunger tip 130 against the interior surface 112 of the syringe barrel 110.

In some embodiments, a coating 140 may be disposed on the external surface 134 of the plunger tip 130. In certain embodiments, coating 140 may be disposed on the entire external surface 134 of plunger tip 130. In other embodiments, coating 140 may be disposed on only a limited portion of the external surface 134 of the plunger tip 130. In certain embodiments, the coating 140 may be disposed on the external surface 134 such that it is positioned between at least a portion of the external surface 134 of the plunger tip 130 and the interior surface 112 of barrel 110. The coating 140 may be configured to reduce the coefficient of friction between the plunger tip 130 and the interior surface 112 of the syringe barrel 110. Stated another way, the coefficient of friction between the material of the coating 140 and the material of the syringe barrel 110 may be less than the coefficient of friction between the material of the plunger tip 130 and the material of the syringe barrel 110. The coating 140 may be disposed along portions of the plunger tip 130 that would otherwise contact the interior surface 112 of the syringe barrel 110.

Additionally, in some embodiments, the coating 140 may further be disposed on the external surface 134 of plunger tip 130 in such a way that the coating 140 may provide a barrier between the external surface 134 and a substance 15 loaded in the reservoir 114 such that there is no physical contact between the external surface 134 and substance 15.

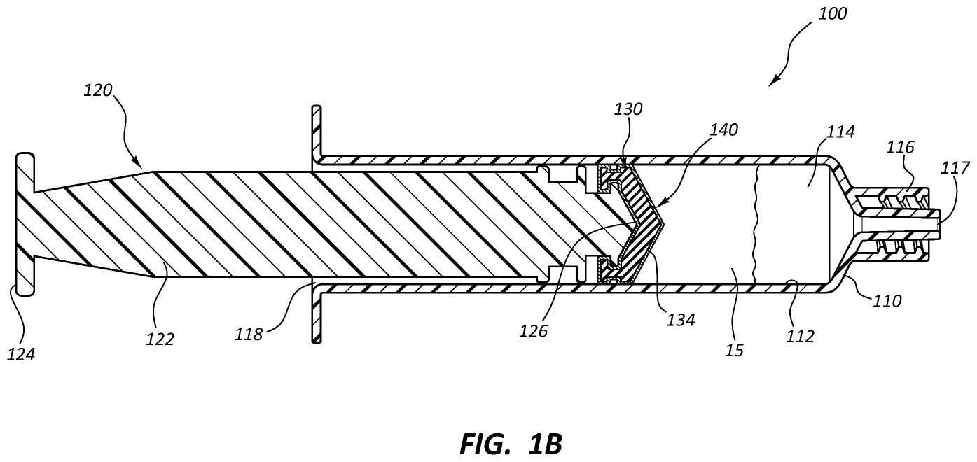

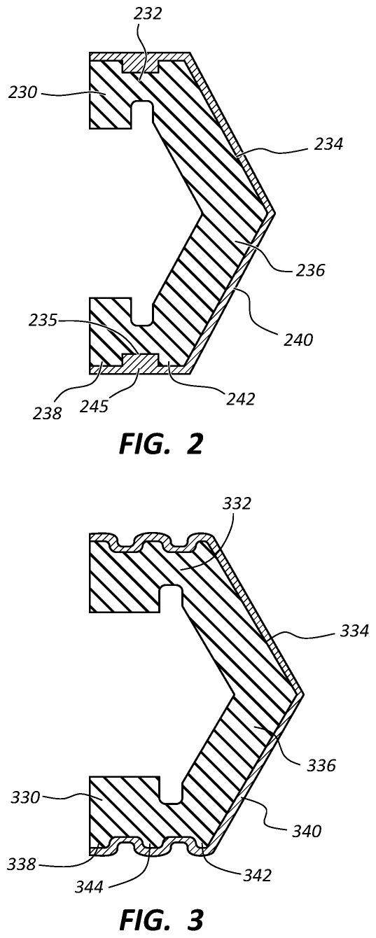

FIGS. 2-8 are a series of views of various embodiments of a syringe assembly and components thereof that can, in certain respects, resemble a syringe assembly and components thereof described in connection with FIGS. 1A and 1B. It will be appreciated that all the illustrated embodiments may have analogous features. Accordingly, like features are designated with like reference numerals, with the leading digits incremented, e.g., to “2,” “3,” or “4.” For instance, the coating is designated as “140” in FIGS. 1A and 1B, and analogous coatings are designated as “240” in FIG. 2, “340” in FIG. 3, and so on. Relevant disclosure set forth above regarding similarly identified features thus may not be repeated hereafter. Moreover, specific features of the syringe assembly and related components (e.g., the coating) shown in FIGS. 1A and 1B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the syringe assemblies and components thereof of FIGS. 2-8. Any suitable combination of the features, and variations of the same, described with respect to the syringe assembly and components thereof illustrated in FIGS. 1A and 1B can be employed with the syringe assemblies and components thereof of FIGS. 2-8, and vice versa. This pattern of disclosure applies equally to further embodiments depicted in subsequent figures and described hereafter.

Referring to FIG. 2, a cross-sectional side view of a component of a syringe assembly is shown, i.e., a plunger tip 230 having an external surface 234. A coating 240 may be disposed on at least a portion of external surface 234. Plunger tip 230 may include an annular wall 232 and a top portion 236 coupled to the annular wall 232. When the plunger tip 230 is part of a syringe assembly (such as shown in FIGS. 1A and 1B), the annular wall 232 may be in contact with an interior surface of a syringe barrel (112 of FIG. 1B). The coating 240 may be disposed to cover all or portions of annular wall 232 and/or top portion 236. Annular wall 232 and top portion 236 may comprise any suitable size and shape for a plunger tip. For example, annular wall 232 may comprise a generally cylindrical or circular shape, and top portion 236 may be, for example, flat or may extend outward from or inward toward annular wall 232.

The annular wall 232 may include one or more ridges or sealing rings protruding from its external surface 234. The one or more ridges may have a generally rounded shape and may protrude outward, for example, from external surface 234 of annular wall 232. In certain embodiments, annular wall 232 may have a generally circular shape, and the one or more ridges may extend around an outer circumference of annular wall 232. As shown, the plunger tip 230 can comprise a proximal ridge 238 disposed around at least a portion of a circumference of the plunger tip 230 at or adjacent a proximal end of the plunger tip 230. The plunger tip 230 can also comprise a distal ridge 242 disposed around at least a portion of the circumference of the plunger tip 230 at or adjacent the distal end of the plunger tip 230. Stated another way, the distal ridge 242 may be disposed distal of the proximal ridge 238.

As also discussed below, the coating 240 may coupled to the plunger tip 230 through adhesion of the coating material to the plunger tip material. That is, the coating 240 may be applied to the plunger tip 230 in a manner in which the coating 240 is coupled or adheres to the plunger tip 230. Additionally or alternatively, the coating 240 and plunger tip 230 may be coupled through use of secondary adhesive materials and/or mechanical fasters or features. For example, the plunger tip 230 and coating 240 may comprise interlocking mechanical features. In the embodiment of FIG. 2, the plunger tip 230 comprises an annular groove or recess 235 that interlocks with an annular ring or protrusion 245 on the coating 240. Other interlocking features are also within the scope of this disclosure. Geometric features with mating portions (such as 235 and 245) may be configured to support or reinforce the coupling of a coating 240 to a plunger tip 230, including in embodiments wherein the coating 240 is configured to adhere to the plunger tip 230.

Referring to FIG. 3, a cross-sectional side view of a coated plunger tip 330 according to another embodiment is shown. Plunger tip 330 may comprise an annular wall 332, a top portion 336, and an external surface 334. As shown, the plunger tip 330 can comprise a proximal ridge 338 and a distal ridge 342 each disposed around at least a portion of the circumference of the plunger tip 330. The plunger tip 330 can further comprise a medial ridge 344 disposed around at least a portion of the circumference of the plunger tip 330 at a position between each of the proximal ridge 338 and the distal ridge 342. In certain embodiments, the plunger tip 330 may comprise one ridge, two ridges, three ridges, four ridges, five ridges, six ridges, or another suitable number of ridges. As illustrated, coating 340 may conform to the shape of the external surface of these features.

As also noted above, one or more the ridges 342, 344, 338 may be configured to be compressed or otherwise deformed to facilitate sealing of the plunger tip 330 against an interior surface of a syringe barrel (112 of FIG. 1B). Annular recesses may be disposed adjacent one or more the ridges 342, 344, 338 and may be configured to accommodate deformation of the ridge. Thus, annular recesses may work in combination with the ridges 342, 344, 338 to facilitate sealing. Furthermore, as shown in FIG. 3, embodiments wherein a coating 340 follows the contours of these features, the coating may be configured to allow or accommodate deformation of the ridges 342, 344, 338 into the annular recesses. Stated another one, the contour and thickness of the coating 340 may allow the ridges 342, 344, 338 to deform in a functionally equivalent or similar way to the deformation of the ridges of an uncoated plunger tip.

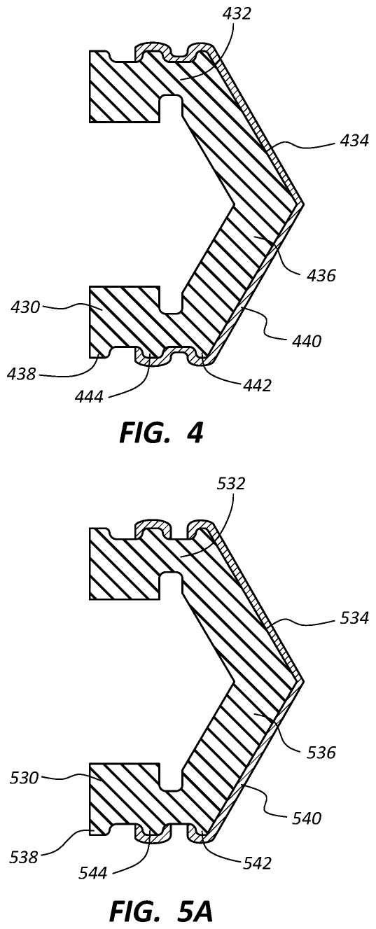

In some embodiments, a coating may be disposed mainly on certain parts of annular wall. For example, a coating may be disposed on one or more of the ridges. This may be useful where the plunger tip is shaped so that certain ridges interact with and/or generate a significant portion of the friction associated with interaction between the plunger tip and the interior surface of the syringe barrel. FIG. 4 shows a cross-sectional side view of a coated plunger tip 430 according to an embodiment in which a coating 440 is disposed on the top portion 436 and includes the distal ridge 442 and the medial ridge 444, but not the proximal ridge 438.

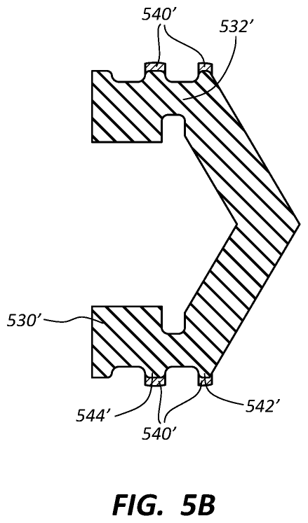

In accordance with the foregoing, in some embodiments a coating may be further restricted to parts of the annular wall or features that contact the interior surface of the syringe barrel. FIG. 5A shows a cross-sectional side view of a coated plunger tip 530 according to an embodiment in which a coating 540 is disposed mainly on the top portion 536 and on the distal ridge 542 and the medial ridge 544, but is absent from the proximal ridge 538 and other parts of the annular wall 532.

Still further, in some embodiments within the scope of this disclosure, the coating may only be disposed on one or more ridges, and absent from other portions of the plunger tip. For example, FIG. 5B illustrates an example of a plunger tip 530′ with a coating 540′ disposed on annular ridges 544′ and 542′ wherein the remainder of the plunger tip 530′ is uncoated. In some embodiments the coating 540′ may only be disposed on a portion of the ridges 542′, 544′, including embodiments wherein the coating is only disposed on a radially outward-most portion of the ridges 542′, 544′. For example, as shown in FIG. 5B, the coating may only be disposed on a top surface of the ridges 542′, 544′, meaning the radially outward-most portion of the ridges 542′, 544′. Embodiments wherein the coating 540′ also extends down the sides of the ridges (for example as shown in ridge 544 of FIG. 5A) are also within the scope of this disclosure. The coating may be disposed on portions of the annular wall 532′ that generate a substantial amount of the friction between the plunger tip 530′ and the interior surface of a syringe barrel (112 of FIG. 1B).

As mentioned above, syringe tips with any number of ridges are within the scope of this disclosure. Coatings of any of the types described above may be used in connection with any number of ridges. For example, coatings positioned on the tops of the ridges, such as shown in FIG. 5B with coating 540′, may be disposed on any number of ridges, including embodiments wherein any subset of the total ridges on the plunger tip include coatings. Furthermore, combinations, such as configurations where certain ridges are coated only on the tops of the ridges (such as shown in FIG. 5B) and certain ridges are coated on the top and sides of the ridges (such as shown on ridge 544 of FIG. 5A) are likewise within the scope of this disclosure. Additionally, combinations wherein certain ridges are coated and certain adjacent annular recesses (such as shown between ridges 442 and 444 of FIG. 4) are also within the scope of this disclosure. In other words, any arrangement of coating for any specific ridge, can be in the form of any of the examples shown herein, in connection or combination with any other arrangement of coating shown herein.

The syringe barrel of any of the embodiments of the present disclosure may comprise any size, shape and material known in the art to be suitable for such syringe components. Accordingly, the syringe barrel may have a cylindrical shape and may be configured to have a plunger disposed within an interior reservoir of the syringe barrel. In some embodiments, the syringe barrel may comprise a plastic or polymeric material.

The handle portion of the plunger of any of the embodiments of the present disclosure may comprise any size, shape and material known in the art to be suitable for such syringe components. The handle portion may comprise a stiff or rigid material that can transfer a force exerted upon it to a plunger tip to which it is coupled or attached to move the plunger tip within the syringe barrel. In some embodiments, the handle portion may comprise a plastic or polymeric material. The handle portion may be longer in its length than in its width or diameter such that it can extend through the entire length of a syringe barrel. Such an elongate design allows the handle portion to be used to move a plunger tip throughout the entire length of a syringe barrel.

The plunger tip may be a tip that is integrally molded to the plunger handle portion or a tip that is attached separately. The plunger tip may be of any design that is of a suitable size and shape for being disposed or otherwise inserted into a syringe barrel such that the plunger tip is in mechanical communication with an interior surface of the syringe barrel. For example, the plunger tip may have a generally cylindrical or circular shape, and the syringe barrel may have a generally cylindrical or circular shape such that they are in contact with each other along all or a portion of an outer circumference of the plunger tip and an inner circumference of the syringe barrel. Further, the plunger tip may comprise an entirely solid structure or may comprise a structure that is hollow or includes a cavity.

In some embodiments, the plunger tip may comprise a material that is resiliently compressive such that the plunger tip may exert a force against the interior surface of the syringe barrel. Thus, the plunger tip may be capable of compressing and deforming against the interior surface of the syringe barrel when disposed within the interior of the syringe barrel. The force exerted by the resiliently compressive material deforming against the interior surface may serve to hold the plunger tip in position within the syringe barrel until displaced by a practitioner. The force may also be a sealing force that prevents any leakage or transmission past the plunger tip of a substance loaded within a reservoir defined by an interior surface of the syringe barrel. In some embodiments, the plunger tip may comprise a thermoplastic elastomer. In some embodiments, the plunger tip may comprise a material containing silicon, such as, for example, silicone or silicone rubber.

Any of the components of the syringes disclosed herein (e.g., the barrel, plunger, handle portion and/or plunger tip) may comprise any polymeric material, such as, for example, acrylonitrile-butadiene-styrene polymer, polycarbonate, polypropylene, or cyclo-olefin polymer or copolymer. In some embodiments, any of the syringe components disclosed herein, including the plunger tip, may comprise a material that contains silicon, such as, for example, silicone or silicone rubber. Alternatively, any of the syringe components disclosed herein may comprise a silicon-free material. Additionally, any of the syringe components disclosed herein may be lubricated with a silicon-free lubricant.

The coating according to the present disclosure may comprise a material that is deposited onto the plunger tip by, for example, dip coating, sputtering, spray coating, or spin coating techniques. In some embodiments, electrospraying or electrospinning techniques may be used. In some embodiments, the coating may be cross-linked or otherwise bonded to the material of the plunger tip. In some embodiments, the coating may have a thickness from about 0.1 μm to about 50 μm, or from about 0.5 μm to about 1.5 μm.

The coating according to the present disclosure may comprise a different material than that of the plunger tip. In some embodiments, the material properties of the materials used for the coating and the plunger tip may differ in certain respects. Accordingly, the coating may comprise any suitable material including those that provide a reduced-friction interface, as compared to the plunger tip material, between a plunger tip and an interior surface of a syringe barrel. Thus, the coating may comprise a material that provides a lower coefficient of friction between the coating and the interior surface of the syringe barrel than the coefficient of friction between the plunger tip and the interior surface of the syringe barrel.

In some embodiments, the coatings of the present disclosure may also comprise a material that isolates a plunger tip from a substance loaded into a reservoir defined by an interior surface of a syringe barrel by preventing physical contact between the plunger tip and the substance. Thus, the coating may comprise a material that is impermeable to either the substance or to a material contained in the plunger tip. For example, the coating may prevent a silicon-containing material, such as silicone, of a plunger tip from contaminating a substance comprising PVA or gelatin foam loaded in the syringe.

The coating material may be formulated so as to be capable of adhering or bonding to the plunger tip material. In some embodiments, the coating may comprise one or more polymeric materials. For example, the coating may comprise polyolefins, polyethylene, perfluoropolyether (PFPE), or perfluoropolyalkylether (PFAE). Polyethylene coatings may comprise LLDPE, HDPE, ePTFE, PTFE, or a composite of any of these materials. In certain embodiments, the plunger tip may comprise silicone and the coating disposed on the plunger tip may comprise LLDPE, HDPE, ePTFE, PTFE, or a composite of any of these materials.

Methods for manufacturing a syringe assembly are also provided by the present disclosure. Referring again to FIGS. 1A and 1B, one embodiment of such a method may comprise applying a coating 140 to a plunger tip 130. Such applying may comprise depositing a material onto at least a portion of any external surface 134 of the plunger tip 130 such that the deposited material forms the coating 140. For example, the material may be deposited by electrospinning or rotational spinning the material onto the external surface 134 of plunger tip 130. In some embodiments, LLDPE, HDPE, PTFE, or other polymers may be electrospun or rotational spun onto a plunger tip 130 comprising, for example, silicone.

Methods for isolating a substance loaded in a syringe from one or more syringe components are also provided by the present disclosure. Such methods may prevent a syringe component from contaminating or otherwise contacting or interfering with the substance, and vice versa. Referring to FIGS. 1A and 1B, one embodiment of such a method involves isolating a substance 15 from a syringe plunger tip 130. The methods may include applying a coating 140 to an external surface 134 of a syringe plunger tip 130. The coating 140 may be applied via any of the deposition methods described herein. The methods may further include disposing the plunger tip 130 with its coating 140 within a reservoir 114 defined by an interior surface 112 of a syringe barrel 110. The plunger tip 130 may be disposed by, for example, inserting it into the reservoir 114 through an opening 118 in syringe barrel 110. The methods may further comprise loading the reservoir 114 with a substance 15. The substance may be loaded in the reservoir 114, e.g., through an opening 117 in a tip 116 of the syringe barrel 110 that is in communication with the reservoir 114.

When plunger tip 130 is disposed in the reservoir 114, and substance 15 is loaded in the reservoir 114, coating 140 may provide a barrier between the plunger tip 130 and the substance 15. In such a case, coating 140 isolates the substance 15 from plunger tip 130 by preventing direct contact between the material of the plunger tip 130 and the substance 15. In addition, when the plunger is disposed in the reservoir 114 as described, coating 140 may isolate the substance from other syringe components as well (e.g., a plunger 120). In some embodiments, the substance may comprise PVA or gelatin foam, the plunger tip 130 may comprise silicone, and the coating 140 may comprise LLDPE, HDPE, ePTFE, PTFE, or a composite of any of these materials.

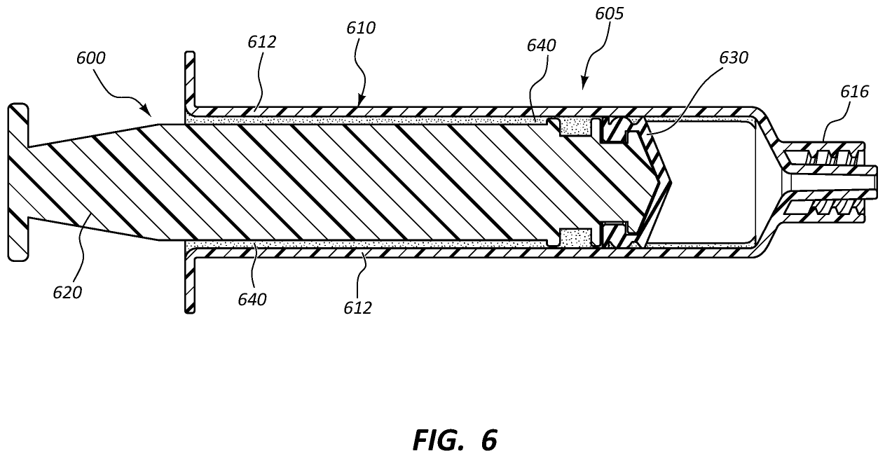

In some embodiments, a syringe system can comprise a barrel having a coating as described herein disposed on at least a portion of its interior surface. FIG. 6 is a cross-sectional side view of an embodiment of a syringe system 600. As depicted, the syringe system 600 can comprise a syringe body 605 comprising a barrel 610 and a plunger 620. The syringe system 100 can also comprise an inlet/outlet port 128 disposed at a distal end of the syringe body 105. As shown, the plunger 620 can comprise a plunger tip 630 coupled thereto. The barrel 610 may include an interior surface 612 and a coating 640 disposed on at least a portion of the interior surface 612. In some embodiments, the coating may be cross-linked or otherwise bonded to the interior surface 612.

The coating according to the present disclosure may comprise a material that is deposited onto the interior surface of the barrel by, for example, dip coating, sputtering, spray coating, or spin coating techniques. In some embodiments, electrospraying or electrospinning techniques may be used. In some embodiments, the coating may be cross-linked or otherwise bonded to the material of the interior surface. In some embodiments, the coating may have a thickness from about 0.1 μm to about 50 μm, or from about 0.5 μm to about 1.5 μm.

The coating according to the present disclosure may comprise a different material than that of the interior surface of the barrel. In some embodiments, the material properties of the materials used for the coating and the interior surface may differ in certain respects. Accordingly, the coating may comprise any suitable material including those that provide a reduced-friction interface, as compared to the interior surface material, between a plunger tip and the coated interior surface. Thus, the coating may comprise a material that provides a lower coefficient of friction between the plunger tip and the coating than the coefficient of friction between the plunger tip and the material of the interior surface without the coating.

The coatings of the present disclosure may also comprise a material that isolates the interior surface of the barrel from a substance loaded into the syringe barrel by preventing direct contact between the interior surface and the substance. Thus, the coating may comprise a material that is impermeable to the substance, and thereby reduce or eliminate the permeability of the interior surface for the substance. Coatings described herein may be used to reduce the permeability of syringe barrels or other components (e.g., stopcocks) comprising polycarbonates or other permeable materials.

The coating material may be formulated so as to be capable of adhering or bonding to the material of the interior surface. In some embodiments, the coating may comprise one or more polymeric materials. For example, the coating may comprise polyolefins, polyethylene, perfluoropolyether (PFPE), or perfluoropolyalkylether (PFAE). Polyethylene coatings may comprise LLDPE, HDPE, ePTFE, PTFE, or a composite of any of these materials. In some embodiments, the coating may comprise a thermoplastic elastomer. Thermoplastic elastomers for such use may comprise, without limitation, styrenic block copolymers, polyester-ester block copolymers, polyether-ester block copolymers, polyurethanes, polyamides or combinations thereof.

In some embodiments, the coating 640 may be configured to isolate the material of the syringe barrel 610 from materials within the reservoir of the syringe barrel 610. The coating 640 may be less permeable than the material of the syringe barrel 610 and may prevent or reduce leaching of materials between the contents of the syringe barrel 610 and the material of the syringe barrel 610 and vice versa.

Further, in various embodiments the use of a coating as disclosed herein may reduce or eliminate the need to use a lubricant (e.g., a lubricant containing silicone) within the syringe assembly (e.g., within the syringe barrel), which, in turn, may prevent such a lubricant from contaminating or otherwise affecting a substance held within the syringe. For example, in some instances silicone lubricants may coat or otherwise interfere with the hydration of PVA or gelatin foams. Coatings used in place of silicone lubricant may therefore be configured for use with PVA or gelatin foams. Additionally or alternatively, in certain embodiments, a coating as disclosed herein may be used to achieve a specific breakaway force with respect to the movement of a plunger tip through a syringe barrel. This breakaway force may be between 0.5 lbf and 3 lbf, 1 lbf and 2 lbf, less than 3 lbf, less than 2 lbf, less than 1.5 lbf, or less than 1 lbf. In other embodiments, a coating as disclosed herein may configured such that coefficients of static and dynamic friction between the coating and a surface of the syringe assembly (e.g., the interior surface of the syringe barrel) are closely matched, for example within 5% of each other, which may facilitate smooth or even use of the syringe.

In any of the foregoing embodiments, the syringe assembly and components thereof may be sterilization compatible materials. “Sterilization compatible materials,” as used herein, refers to materials capable of being sterilized without rendering the materials unsuitable for their intended purposes. If a material is configured for sterilization by at least one method of sterilization without being rendered unsuitable for its intended purpose, then the material is a “sterilization compatible material.” For example, a polymeric barrel may deform when autoclaved at temperatures sufficient to sterilize the barrel, rendering the barrel unsuitable for its intended purpose of maintaining a seal with a cylindrical or circular plunger. However, if the same polymeric barrel may be sterilized by another sterilization technique, such as irradiation, and maintain suitability for its intended purpose, then the polymeric material is a “sterilization compatible material.”

In any of the foregoing embodiments, the syringe assembly and components thereof may be made from irradiation compatible materials. “Irradiation compatible materials,” as used herein, refers specifically to materials capable of being sterilized by irradiation without rendering the materials unsuitable for their intended purposes. For example, a plunger or interior surface of a barrel may comprise a material or lubricant that upon irradiation changes in physical properties such that the syringe is unsuitable for its intended purpose. For example, irradiation may alter certain materials or lubricants such that a syringe using those materials would have an unacceptably high initial peak force required to start movement of the plunger. Or, in another example, irradiation may alter certain materials or lubricants such that a syringe using those materials would have an unacceptably non-uniform force required for travel of the plunger over the length of the barrel or an unacceptably high force required for travel of the plunger over the length of the barrel.

The coating, syringe plunger tip, and other components disclosed herein may be used with a syringe loaded with any suitable substance for loading into a syringe. In certain embodiments, the syringe may be loaded with a substance comprising an embolic agent comprising a microparticle and/or microsphere. Examples of microparticles include polyvinyl alcohol (PVA) microparticles. Examples of microspheres include trisacryl cross-linked with gelatin microspheres, sodium acrylate vinyl alcohol copolymer microspheres, and polyvinyl alcohol based hydrogels.

While specific embodiments of coatings for syringes and methods related thereto have been described, it is to be understood that the disclosure provided is not limited to the precise configuration and components disclosed. Various modifications, changes, and variations apparent to those of skill in the art having the benefit of this disclosure may be made in the arrangement, operation, and details of the devices, methods, and systems disclosed, with the aid of the present disclosure.

Furthermore, any reference to “one embodiment,” “an embodiment,” or “the embodiment,” as used throughout this disclosure, means that a particular feature, structure, or characteristic described in connection with that embodiment is included in at least one embodiment. Thus, the quoted phrases, or variations thereof, as recited throughout this specification are not necessarily all referring to the same embodiment.

Without further elaboration, it is believed that one skilled in the art can use the preceding description to utilize the present disclosure to its fullest extent. The examples and embodiments disclosed herein are to be construed as merely illustrative and exemplary and not as a limitation of the scope of the present disclosure in any way. It will be apparent to those having skill in the art, and having the benefit of this disclosure, that changes may be made to the details of the above-described embodiments without departing from the underlying principles of the disclosure herein.

1. A syringe comprising:
a barrel comprising an interior surface;
a plunger comprising:
an elongate handle portion defining a proximal end and a distal end; and
a plunger tip coupled to the distal end of the elongate handle portion, the plunger tip comprising a first material and including an annular wall and a top portion coupled to the annular wall; and
a coating disposed on at least a portion of an external surface of the plunger tip, the coating comprising a second material,
wherein at least one material property of the first material differs from at least one material property of the second material.

2. The syringe of claim 1, wherein the second material comprises polyethylene, perfluoropolyether (PFPE), or perfluoropolyalkylether (PFAE).

3. The syringe of claim 2, wherein the second material comprises linear low-density polyethylene (LLDPE), high-density polyethylene (HDPE), expanded polytetrafluoroethylene (ePTFE), polytetrafluoroethylene (PTFE), or composites thereof.

4. The syringe of claim 1, wherein a coefficient of friction between the second material and the interior surface is lower than a coefficient of friction between the first material and the interior surface.

5. The syringe of claim 1, wherein the coating isolates the first material from a substance loaded in the reservoir by preventing physical contact between the first material and the substance.

6. The syringe of claim 5, wherein the plunger tip comprises silicone and the substance comprises polyvinyl alcohol (PVA) or gelatin foam.

7. The syringe of claim 1, wherein the coating is absent from the external surface of a portion of the annular wall.

8. The syringe of claim 1, wherein the syringe is free of liquid silicone lubricants.

9. The syringe of claim 1, wherein the coating has a thickness from about 0.1 μm to about 50 μm.

10. The syringe of claim 1, wherein the coating is adhered to at least a portion of an external surface of the plunger tip.

11. The syringe of claim 1, wherein the coating is covalently bonded to the portion of the external surface of the plunger tip.

12. The syringe of claim 1, wherein the plunger tip and coating comprise a geometric coupling feature.

13. A syringe component comprising:
a plunger tip comprising a first material, and including an annular wall and a top portion coupled to the annular wall;
a coating disposed on at least a portion of an external surface of the plunger tip, the coating comprising a second material,
wherein at least one material property of the first material differs from at least one material property of the second material.

14. The syringe component of claim 13, wherein a coefficient of friction between the second material and an interior surface of a syringe is lower than a coefficient of friction between the first material and the interior surface of the syringe.

15. The syringe component of claim 13, wherein the coating is configured to isolate the first material in the plunger tip from a substance loaded in a reservoir defined by an interior surface of a syringe by preventing physical contact between the first material and the substance.

16. A method of manufacture of a syringe assembly, comprising applying a coating to a plunger tip,
wherein the plunger tip comprises a first material, the coating comprises a second material, and
wherein at least one material property of the first material differs from at least one material property of the second material.

17. The method of claim 16, wherein applying the coating to the plunger tip comprises depositing the second material onto an external surface of the plunger tip.

18. The method of claim 17, wherein depositing comprises dip coating, sputtering, spray coating, or spin coating the second material onto the external surface.

19. The method of claim 16, further comprising covalently bonding the second material to the external surface.

20. The method of claim 16, further comprising coupling the plunger tip to a syringe plunger.
